(19) United States
(12) Patent Application Publication
(10) Pub. No.: US 2011/0082543 A1
(43) Pub. Date: Apr. 7, 2011

(54) Methods and Devices for Preventing or Delaying Posterior Capsule Opacification

(75) Inventors: David B. SOLL, Ambler, PA (US); Ihab L. KAMEL, Drexel Hill, PA (US); Inez Amina Ruiz-White, Mount Laurel, NJ (US)

(73) Assignee: CLEO COSMETIC AND PHARMACEUTICAL COMPANY, LLC, Ambler, PA (US)

(21) Appl. No.: 12/899,093

(22) Filed: Oct. 6, 2010

Related U.S. Application Data
(60) Provisional application No. 61/249,000, filed on Oct. 6, 2009.

(51) Int. Cl. A61F 2/16

(52) U.S. Cl. 623/6.38; 623/6.62

(57) ABSTRACT

Several methods for preventing, minimizing, or delaying the incidence of posterior capsule opacification are provided. A first method involves chemically activating the surface of an implantable ocular device, such as an intraocular lens or a capsular tension ring, by grafting a chemical moiety onto the surface of the device, covalently attaching a non-cytotoxic inhibitor compound to the chemical moiety to produce an inhibitor implantable ocular device, and implanting this inhibitor implantable ocular device into the capsular bag of an eye of a patient during extracapsular cataract surgery. Appropriate inhibitor compounds include RGD mimetics, RGD peptides, and flavonoids. A second method involves surface modifying the exterior surface of a capsular tension ring by covalently attaching a mitotic inhibitor, preferably a conjugate of methotrexate and a bovine serum albumin, and implanting this inhibitor tension ring into the capsular bag of an eye of a patient during extracapsular cataract surgery. A third method involves surface modifying the exterior surface of a capsular tension ring by coating or grafting the exterior surface with a charged polyethylamine and implanting this inhibitor tension ring into the capsular bag of an eye of a patient during extracapsular cataract surgery. An implantable ocular device according to the invention, such as an intraocular lens or a capsular tension ring, contains a substrate with a chemical moiety grafted thereon and a non-cytotoxic inhibitor compound covalently bonded to the chemical moiety or contains a substrate modified with a mitotic inhibitor or charged polyethylamine. The inhibitor devices inhibits proliferation and migration of lens epithelial cells on the posterior capsule of the eye of the patient, thereby preventing, minimizing, or delaying the onset of posterior capsule opacification.

### CROSS-REFERENCE TO RELATED APPLICATIONS

This application claims priority to U.S. Provisional Patent Application No. 61/249,000, filed Oct. 6, 2009, the disclosure of which is herein incorporated by reference in its entirety.

### BACKGROUND OF THE INVENTION

Posterior Capsular Opacification (PCO), also known as a secondary cataract or after cataract, is the opacification of the posterior lens capsule following extracapsular cataract surgery. It is a major complication of cataract and intraocular lens surgeries and affects 40-50% of people within two years of cataract extraction. This condition clouds the vision of millions of people and can result in gradual vision loss.

Cataracts are part of the normal aging process and more than half of all Americans over the age of 65 have a cataract. Currently, there are several operative procedures for removing cataracts: intracapsular cataract extraction, extracapsular cataract extraction, and phacoemulsification. In extracapsular cataract extraction, the cataractus material is expressed from the eye through a moderately large incision. Phacoemulsification, a form of extracapsular cataract surgery, is, at present, the most common method of cataract removal in the United States and in many Western countries. In phacoemulsification, which is performed through a small incision, the cataractus lens material is ground up by ultrasonic energy and aspirated from the eye by suction. In contrast, in intracapsular cataract extraction, the entire cataract is removed from the eye in one piece. There are more retinal complications associated with intracapsular surgery, and this form of cataract surgery does not lend itself as readily to intraocular lens implantation as do the extracapsular procedures.

In all types of extracapsular surgeries, a central disc of the anterior lens capsule is removed and the peripheral portion of the anterior lens capsule, along with the entire posterior portion of the lens capsule, is left in the eye. Most often, an intraocular lens (IOL) is inserted into the remaining capsular bag of the patient's eye at the time of the cataract removal. This synthetic lens has clear optics and replaces the removed cataractus lens material.

The majority of IOLs have two components: a central lens, typically with a diameter of about 5 to 7 mm, and supporting loops or arms, known as haptics, which are typically 180° apart. These haptics may be an integral part of the lens and of the same material as the lens, or of a different material than the optical portion of the lens but attached to this optical portion. For example, haptics may be made of a flexible material such as PMMA (polymethylmethacrylate), polypropylene, or any other inert biocompatible, flexible material. They function to stabilize the optical portion of the lens, keeping it centrally located.

Most synthetic intraocular lenses are inserted behind the iris with the optic of the lens resting on the posterior lens capsule or very close to it. A portion of each haptic rests against the equatorial portion of the remaining lens capsule bag.

In many situations, particularly if there is evidence of a segmental zonular deficiency, missing or damaged zonules, lens subluxation, myopia, pseudo-exfoliation, zonulolysis, or Marfan's syndrome, a capsular tension ring is inserted into the capsular bag following removal of the cataractous lens material. When inserted, this thin, flexible ring exerts tension or pressure on the equatorial portions of the capsular bag and maintains the symmetrical shape of the capsular bag, even in areas of zonular deficiency, thus stabilizing and re-centering the bag. Capsular tension rings, also known as capsular rings and capsular tension segments, for example, are typically formed of flexible materials, such as PMMA (polymethylmethacrylate).

Capsular tension rings exert mild pressure in the equatorial portion of the capsular bag, the region of the bag where residual lens epithelial cells (LECs) are known to proliferate. Eventually, such proliferation and migration of lens cells across the available surfaces of the capsular bag and the capsular tension ring to the central surface of the posterior capsule lead to posterior capsule opacification that adversely affects vision.

Additionally, there are always residual lens epithelial cells (LECs) which remain attached to the remaining portions of the anterior capsule and to the equatorial portions of the lens capsule at the conclusion of the surgical procedure. These remaining lens epithelial cells reproduce and migrate to the posterior portion of the lens capsule along the available surfaces of the capsular bag. The abnormal accumulation of LECs on the posterior capsule can form clusters called Elschnig's pearls, which haze the capsule. The LECs can also differentiate into myofibroblasts and form a white, fibrous membrane on the posterior capsule, also resulting in opacification. Thus, it is the formation of Elschnig's pearls and the metaplasia of LECs which are together responsible for the opacification of the posterior capsule. This opacification can in turn cause significant visual loss and, if severe and advanced, even blindness.

The rate of occurrence of PCO is associated with the surface properties of the IOL. Currently, the primary manufactured IOLs are fabricated from polymethylmethacrylate (PMMA), silicone, and soft acrylic. Significant differences in the frequency of occurrence of PCO among the lens materials have been found: soft acrylic (10%), silicone (40%), and PMMA (56%) (Hollick et al.; Ophthalmology; 106:49-55 (1996)). Studies have demonstrated that the surface properties of an IOL can mediate the inhibition of LEC growth and such inhibition does not necessarily involve an intracellular mechanism (Nagata; J. Cataract Refract. Surg.; 24:667-673 (1998); Oshika; Br. J. Ophthalmol.; 82:549-553 (1998)).

One reason why soft acrylic lenses are associated with lower incidences of PCO may be due to their ability to bind to fibronectin and inhibit further LEC growth (Linnola; Academic Dissertation for the Department of Ophthalmology and Department of Medical Biochemistry, University of Oulu (2001)). Specifically, LECs bind to the surface of acrylic lenses and form a monolayer of cells between the IOL and the capsular bag. Attachment of LECs to the surface of the IOL is mediated by fibronectin and laminin binding. The concept of simultaneous LEC binding to the IOL and the lens capsule at the same time to form a sandwich pattern is referred to as the “sandwich theory.” Accordingly to this theory, IOLs containing a bioactive surface that promotes fibronectin and laminin binding will result in a sandwich pattern of LEC attachment. This sandwich structure, when it occurs, is, formed by the monolayer of LECs between the IOL and capsular bag, hinders epithelial growth, and results in a clinically clear posterior capsule (Linnola, 2001).

A variety of different methods have been developed to remediate PCO. These methods include capsule polishing to remove residual lens epithelial cells, various types of surface modified IOLs, edge modification of the IOL to prevent cells from growing underneath the IOL, and antimetabolic agents and immunotoxins which have been injected into the anterior segment and under the IOL.

For example, in animal experiments, cytotoxic drugs administered during surgery or implantation have been investigated as a means of inhibiting the growth of lens epithelial cells. An example of such a drug is methotrexate (MTX), as described in U.S. Pat. No. 4,515,794. MTX kills dividing cells preferentially, though not exclusively, and is used in cancer chemotherapy.

As described in the '794 patent, MTX has been used as a mitotic inhibitor by instilling a solution containing a specific concentration of methotrexate into the anterior chamber of the eye after lens removal. The solution containing MTX is osmotically balanced to provide minimal effective dosage when instilled into the anterior chamber of the eye, thereby inhibiting subcapsular epithelial growth.

However, because MTX is not specific as to the type of cell that it kills, serious side effects can occur. Moreover, epithelial cells must divide for MTX to exert its cytotoxic effect. The drug should therefore remain in the eye at least through the generation time of the lens epithelial cells. While these cells normally divide very slowly and only at the equator, division is stimulated by injury, such as would occur during surgery, occurring within 48 hours. Mitotic inhibitors comprised simply of solutions of methotrexate or other cytotoxins which are instilled in the aqueous fluid of the eye would be continually diluted by inflow of aqueous fluid which is renewed with a half time of about three hours. This dilution in turn decreases the ability of the drug to inhibit growth of the epithelial cells which remain after lens extraction.

A method for preventing PCO which utilizes a targeted mitotic inhibitor is described in U.S. Pat. No. 4,918,165, the disclosure of which is herein incorporated by reference 1. In this method, a cytotoxic agent coupled with means to target the cytotoxic agent to particular cell types is instilled into the anterior or posterior chamber of the eye during or immediately following cataract surgery. Preferably, this is accomplished by coupling methotrexate with an antibody, such as anticollagen, thus yielding a conjugate in which the molar ratio of methotrexate to anticollagen is about 1:1 to 10:1.

Targeting of the MTX allows for a much lower concentration of MTX (compared to the use of free MTX as in U.S. Pat. No. 4,515,794) to be instilled into the eye's anterior or posterior chamber, thus decreasing the possibility that the patient will experience harmful side effects. Furthermore, a larger concentration of MTX can also be used, as needed, with the likelihood of side effects occurring also being lessened due to the targeting of the MTX specifically to lens epithelial cells. The '165 patent also describes the surface modification of IOLs with mitotic inhibitors. These surface modified IOLs are attractive because they are stable and lack general toxicity. However, production of these IOLs requires an extra manufacturing step and such IOLs are effective only when cells are in contact with the surface.

When a significant amount of opacification occurs on the posterior capsule, the only effective treatment is to make an opening in the posterior capsule to allow light and images to pass through the eye to the retina, enabling the patient to see clearly again. However, making a surgical opening in the posterior capsule (surgical capsulotomy) is an invasive procedure which may result in post-operative infections and other complications, and is typically only performed in areas where a Nd:YAG laser is not available.

Currently, Nd:YAG laser capsulotomy is the most common effective treatment for PCO. In this method, a Nd:YAG laser is used to generate infrared light impulses which create tiny openings in the lens capsule through photodisruption. These openings allow light, which was blocked by the haze of the opacification, to pass through the membrane to the retina. A special ophthalmic YAG laser is necessary to perform this procedure. Nd:YAG laser capsulotomies are very expensive and cost Medicare over $250 million annually due to the combination of expensive lasers and medical personnel services. They are the second-most frequently performed surgical procedure for Medicare beneficiaries, second only to cataract surgeries.

Since YAG laser capsulotomies are not invasive procedures, intraocular infections do not typically occur after these treatments. However, there are significant complications that can occur following any type of posterior capsulotomy procedure, including Nd:YAG laser capsulotomy. Such complications include elevated intraocular pressure, IOL damage, retinal detachment, cystoid macular edema, iris hemorrhage, uveitis/vitritis, and reopacification. Treatments for complications associated with YAG laser capsulotomy only add to the major financial implications of PCO.

Since patients undergoing cataract surgery are at high risk of developing PCO, everyone over the age of 65 is at risk for PCO. It is estimated that about 480,000 people will be treated for PCO in the United States every year. Based on the 2000 U.S. Census, there are 35 million people over the age of 65, representing 12% of the total population. Clearly, the societal and human cost of PCO is profound and as the population ages, this problem (and its associated costs) will increase approximately 3% per year (Market Scope, 2004). It is estimated that by 2050 there will be 85 million people in the United States who are 65 years or older, representing 21% of the population (US Census, 2001).

None of the PCO remediation methods which has been developed has been satisfactory or is currently being routinely employed in a clinical setting. Accordingly, there remains a need in the art for a method for eliminating or dramatically reducing the need for PCO treatments and the associated costs by preventing or delaying the onset of this debilitating condition. Prevention of PCO formation will also cause a decline in the number of Nd:YAG laser procedures and therefore reduce the financial burden associated with posterior capsulotomies. Prevention of the need for a posterior capsulotomy will also have significant medical benefits by eliminating the medical and surgical complications that are associated with the posterior capsulotomy procedure.

### SUMMARY OF THE INVENTION

A method for preventing, minimizing or delaying posterior capsule opacification comprises:
(a) chemically activating a surface of an implantable ocular device by grafting a first chemical moiety onto the surface of the device;
(b) covalently attaching a first non-cytotoxic inhibitor compound to the first chemical moiety on the chemically activated surface of the implantable ocular device to produce an inhibitor implantable ocular device; and
(c) implanting the inhibitor implantable ocular device into a capsular bag of an eye of a patient during extracapsular cataract surgery.

A second method for preventing, minimizing or delaying posterior capsule opacification comprises:
(a) surface modifying an exterior surface of a capsular tension ring by covalently attaching a mitotic inhibitor to the exterior surface to produce an inhibitor capsular tension ring; and
(b) implanting the inhibitor capsular tension ring into a capsular bag of an eye of a patient during extracapsular cataract surgery.

A third method for preventing, minimizing or delaying posterior capsule opacification comprises:
(a) surface modifying an exterior surface of a capsular tension ring by coating or grafting the exterior surface with a charged polyethylamine to produce an inhibitor capsular tension ring; and
(b) implanting the inhibitor capsular tension ring into a capsular bag of an eye of a patient during extracapsular cataract surgery.

An implantable ocular device according to the invention comprises a substrate having a surface, a first chemical moiety grafted onto the surface, and a first non-cytotoxic inhibitor compound covalently bonded to the first chemical moiety, wherein the implantable ocular device prevents, minimizes, or delays the formation of posterior capsule opacification when implanted into an eye of a patient during extracapsular cataract surgery.

A first type of capsular tension ring according to the invention comprises a substrate having an exterior surface and a mitotic inhibitor covalently attached to the exterior surface, wherein the capsular tension ring prevents, minimizes, or delays the formation of posterior capsule opacification when implanted into an eye of a patient during extracapsular cataract surgery.

A second type of capsular tension ring according to the invention comprises a substrate having an exterior surface and a charged polyethylamine coated on the exterior surface, wherein the capsular tension ring prevents, minimizes, or delays the formation of posterior capsule opacification when implanted into an eye of a patient during extracapsular cataract surgery.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIGS. 1A-1D are schematic diagrams depicting the typical steps in extracapsular cataract surgery for removing a cataract and placing an intraocular lens in a capsular bag; and

FIG. 2 is a schematic drawing depicting the placement of a capsular tension ring in a capsular bag.

### DETAILED DESCRIPTION OF THE INVENTION

The present invention is directed to methods for preventing, minimizing, or delaying the incidence of PCO which utilize surface modified “inhibitor” implantable ocular devices, including specific surface modified capsular tension rings. The term “implantable ocular device” is intended to encompass any device which may be implanted into the eye, such as, but not limited to, an intraocular lens (IOL) and a capsular tension ring. An inhibitor IOL will inhibit the proliferation and/or migration of reproducing lens epithelial cells and block the accumulation of LECs on the central area of the posterior capsule. Similarly, an inhibitor capsular tension ring will prevent the proliferation and therefore the migration of equatorial lens epithelial cells to the central portion of the posterior capsule and the resulting opacification and fibrosis.

### Method of Preparing Inhibitor Intraocular Devices

As explained in more detail below, a first method involves covalently attaching a non-cytotoxic inhibitor compound, such as an RGD mimetic, RGD peptide, or flavonoid, to the surface of an IOL or capsular tension ring, for example, and implanting this inhibitor implantable ocular device into a capsular bag of an eye of a patient during extracapsular cataract surgery. Optionally and preferably, if the device is an intraocular lens that contains one or more haptic, the method may also involve covalently attaching the same or a similar non-cytotoxic inhibitor compound to the surface of the haptic(s).

The first step in the presently claimed first method involves chemically activating the surface of an implantable ocular device to graft an appropriate chemical moiety which will be used for subsequent linking to an inhibitor compound.

If the device is an IOL, it is preferably formed of a material such as a silicone, a soft acrylic, such as HEMA (hydroxyethylmethacrylate), or a hard acrylic, such as PMMA. The terms “soft” and “hard” acrylic are well known and understood in the art. However, it is also within the scope of the invention to utilize another inert, optically clear plastic material which is known or to be developed and which would exhibit properties appropriate for IOL manufacture and implantation.

In another preferred embodiment, the ocular device is a capsular tension ring. Any shape or design of capsular tension ring known in the art or to be developed may be utilized as the tension ring in this embodiment of the invention, including tension rings of varying diameters and thicknesses and rings containing fixation hook(s) or suturing hole(s). It is within the scope of the invention to utilize closed rings, foldable ring, partial rings, segmental rings, and rings having circumferences of any degree, including rings which span 270°, rings which span 360° or nearly 360°, and all rings which span intermediate angles. The term “capsular tension ring” may be understood to include devices which may also be commonly referred to as capsular rings, capsular tension segments, etc. Any similar device which is designed to come into contact with the fornix or internal equatorial portion of the capsular bag is included in the term “capsular tension ring” according to the invention.

Preferably, the capsular tension ring is formed from PMMA, a type of hard or rigid acrylic polymeric material. It is also within the scope of the invention to utilize another inert plastic material which is known or to be developed and which would exhibit similar properties as PMMA and be able to exert the necessary tension in the capsular bag. For example, other acrylic materials, such as copolymers of HEMA and MMA (methylmethacrylate) would also be appropriate. Some flexibility may be achieved by the design and diameter of the capsular tension ring.

The surface chemical activation of the implantable ocular device is preferably performed using radio frequency (RF) plasma as a primary energy source, which first removes surface contaminants and etches the surface of the device by breaking and re-forming covalent bonds via short-lived free radicals. Subsequently, an appropriate organic vapor is introduced into the plasma chamber to graft the specific desired chemical moiety, such as an amino or carboxyl group, to the surface. For example, grafting with acrylic acid will introduce polyacrylic acid chains containing carboxyl groups onto the surface of the device, and grafting with ethylenediamine will introduce amino groups onto the surface of the device. These functional groups are then used in the following step to covalently link the desired inhibitor compound to the device surface using an appropriate catalyst. Exemplary methods of surface modification are described in U.S. Pat. Nos. 5,080,924; 5,260,093; 5,326,584; and 5,578,079, the disclosures of which are herein incorporated by reference. The disclosure of U.S. Pat. No. 4,918,165, which describes the modification of an intraocular lens using a cytotoxin-antibody conjugate, is also herein incorporated by reference.

Thus, in the second step of the method, a non-cytotoxic inhibitor compound, preferably an RGD mimetic, RGD-containing peptide, or flavonoid, is covalently attached to the chemically activated implantable ocular device to form a modified “inhibitor” implantable ocular device which has an immobilized inhibitor compound on its surface. The term “non-cytotoxic” may be understood to refer to any material which does not have a detrimental or toxic effect on cells. Preferably, the flavonoid, RGD mimetic, or RGD-containing peptide is one which has been shown to exhibit superior potency and efficacy in its ability to inhibit proliferation and migration of human lens epithelial cells.

Flavonoids are polyphenolic compounds which can exert various biochemical activities, including inhibition of proliferation (Pianette; Cancer Res; 62:652-655 (2002)). Specifically, (−)-epigallocatechin-3-gallate (EGCG), a member of the flavonoid family, has been shown to inhibit the proliferation of rabbit LECs by a mechanism which is unknown (Huang; Yan Ke Xue Bao; 16:194-198 (2000)). Preferred flavonoids for use in the method are flavan-3-ols, which have a free hydroxyl group, and include (−)-epigallocatechin-3-gallate, (+)-catechin, (−)(−)epigallocatechin, and (−)(−)epicatechin-3-gallate.

RGD peptides are short peptides which contain the Arg-Gly-Asp (RGD) amino acid sequence, the sequence recognized by several integrins, or are based on the molecular design of structures mimicking some fragment of the RGD sequence. RGD mimetics are chemical compounds which are non-peptidic in nature but which exert the same pharmacology as the RGD itself. Mimetics may be cyclic or acyclic structures. RGD mimetics have been shown to inhibit the cell attachment and migration of human LECs (Kojetinsky; Ophthalmolge; 98:731-735 (2001); Ohrazawa; Ophthalmic Research; 37:191-196 (2005)). RGD mimetics also inhibit the attachment of laminin and fibronectin, extracellular matrix (ECM) proteins responsible for the migration and metaplasia of LECs into myofibroblast cells (Ohrazawa 2005).

Preferred RGD mimetics for use in the method of the invention include, but are not limited to GPIIb-IIIa antagonists, such as, for example, tirofiban, amifiben, orbofiban, fradafiban, sibrafiban, and their acid equivalents; vitronectin receptor antagonists, such as, but not limited to indazole, benzodiazepine, and isoxazoline; and α3β1/α5β1 integrin antagonists, such as SF-6,5 and NS-11. It is also within the scope of the invention to utilize a RGD peptide, such as the RGD-containing cyclic peptide eptifbatide. These compounds all have a free amino acid and/or carboxylic acid group suitable for attachment to the chemically activated surface. However, RGD mimetics tend to be more stable and may be preferred.

The specific functional groups which are present on the particular inhibitor compound dictate the preferred method for attachment to the chemically activated device surface. Specifically, if the compound is an RGD mimetic or RGD peptide (and thus contains an amino group), the chemically activated device may be treated with a water-soluble carbodiimide to link with the carboxyl groups on the device via amide links. In contrast, if the inhibitor compound is a flavonoid (and thus contains hydroxyl groups), it is preferred that the chemically activated device be first treated with oxalyl chloride, which activates and converts the carboxyl groups on the device into highly reactive acid chloride groups. On exposure of the flavonoid to the activated surface, the hydroxyl groups on the inhibitor compound react to form an ester.

These methods of treatment are intended to be exemplary, and alternative methods for linking which are known in the art or to be developed would also be appropriate.

It is also within the scope of the invention to utilize an alternative coupling agent to carbodiimide. There are diverse types of coupling agents, such as succinimidyl 4-[N-maleimidomethyl]cyclohexane-1-carboxylate, [p-maleimidophenyl]isocyanate, and cyanogen bromide, which react with different types of functional groups. These coupling agents may be alternatives to or preferred over carbodiimide, depending on the particular functional groups which are used.

In one embodiment, the implantable ocular device is an IOL that comprises at least one haptic, most preferably two haptics which are situated about 180° apart. Such haptics may be of any type, material, or configuration known in the art or to be developed, and may be of the same material as the IOL or of a different material. In such an embodiment, the method further comprises chemically activating the surfaces of the haptic(s) by grafting a chemical moiety onto the surfaces of the haptic(s) and covalently attaching a non-cytotoxic inhibitor compound to the chemically activated surfaces of the haptic(s). In such an embodiment, the chemical moieties attached to the surfaces of the IOL and the haptic(s) may be referred to as the “first” and “second” chemical moieties, respectively. Similarly, the non-cytotoxic inhibitor compounds which are covalently attached to the chemically activated surfaces of the IOL and the haptic(s) may be referred to as the “first” and “second” inhibitor compounds, respectively. Preferably, the chemical moieties and non-cytotoxic inhibitor compounds which are attached to the surfaces of the haptic(s) are the same as those which are attached to the surface of the IOL. That is, the first and second chemical moieties are the same, and the first and second inhibitor compounds are the same.

Preferably, the steps of chemically activating the surfaces of the haptics and the attachment of the inhibitor compounds to the chemically activated surfaces of the haptics are performed simultaneously with the chemical activation and inhibitor attachment to the surface of the IOL.

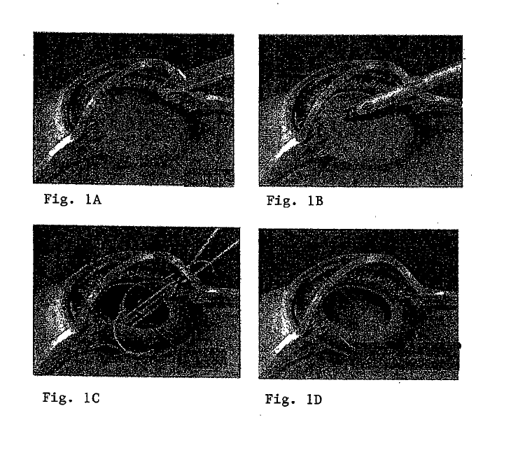

Finally, in a third method step, the modified “inhibitor implantable ocular device” is implanted into a capsular bag of a patient during extracapsular cataract surgery, such as, but not limited to phacoemulsification surgery, using known methods of implantation. A schematic diagram depicting removal of a cataract by extracapsular cataract surgery and implantation of an IOL in a capsular bag is shown in FIGS. 1A-1D. In step A, an incision is made in the eye, followed by removal of the central portion of the anterior lens capsule, breaking up and aspiration of the cataract in step B. The IOL is then implanted into the capsule bag in step C. At the conclusion (step D), the new IOL is in place within the capsular bag.

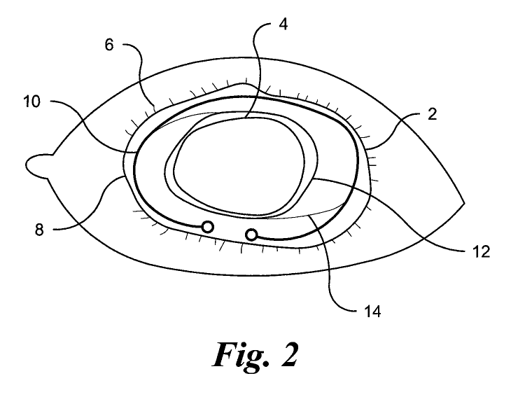

A schematic diagram of a capsular tension ring implanted in a capsular bag is shown in FIG. 2. In this Fig., various critical parts of the capsule bag portion of the eye are shown, including the peripheral edge of the capsular bag 2, the rim of the remaining anterior capsule 4, zonules 6, attached to the periphery of the capsule bag, and areas of missing zonules where the capsule bag has lost its support 8. The capsular tension ring 10 is implanted in the capsular bag, giving it support in the area of the missing zonules and causing the capsule bag to resume its more normal shape. An intraocular lens 12 having a haptic 14 is also present in this diagrammatic capsular bag.

It has been shown that proliferation and migration of residual lens epithelial cells into the visual axis of the posterior capsule causes PCO. Reproducing and proliferating LECs are located in the equatorial region of the lens capsule, exactly where a capsular tension ring is located and exerts an effect. Without wishing to be bound by theory, it is believed that a tension ring having a chemically activated surface which allows covalent attachment of an LEC inhibitor will prevent LEC accumulation at the posterior capsule. That is, attachment of the inhibitor to the tension ring creates a barrier surface that should prevent residual anterior and equatorial LECs from proliferating across the tension ring and accumulating at the posterior capsule.

Without wishing to be bound by theory, it is also believed that an IOL having a chemically activated surface which allows covalent attachment of an LEC inhibitor will prevent LEC accumulation at the posterior capsule. Attachment of the inhibitor to the IOL creates a barrier surface that should prevent residual anterior LECs from proliferating across the IOL and posterior portion of the lens capsule and accumulating at the posterior capsule in a location that will prevent clear and useful vision.

The surface chemically activated capsular tension ring or intraocular lens, for example, to which an RGD mimetic, RGD peptide, and/or flavonoid compound is covalently linked, will come in contact with the residual anterior LECs and is expected to prevent opacification by blocking cellular activities leading to Elschnig's pearl formation, LEC metaplasia, or LEC accumulation across the tension ring or IOL.

Further, it has been found that reproduction and metaplasia of LECs causes PCO and is associated with extracellular matrix proteins. Myofibroblast cells are also responsible for the opacification of the posterior capsule. The differentiation of LECs to myofibroblast cells results in the formation of a white fibrous material on the posterior capsule (Apple; Surv. Ophthalmol; 37:73-116 (1992); Green; Trans. Ophthalmol. Sci UK; 104:727-739 (1998); Saxby; Br. J. Ophthalmol; 82:945-952 (1998)). The binding of ECM proteins, specifically fibronectin, to its integrin receptor is important for the differentiation of LECs to myofibroblasts (Yoshino; IOVS; 39:S307 (1998)). It is believed that blocking fibronectin attachment to its receptor membrane binding site should prevent LECs metaplasia and reduce PCO formation. That is, the RGD mimetic or RGD peptide on the surface of the tension ring or intraocular lens will inhibit the attachment of ECM proteins to their appropriate binding receptors and block the biochemical or physiochemical signaling for cellular migration. The inhibition of cellular migration will prevent anterior LECs from migrating across and accumulating at the posterior capsule and forming Elschnig's pearls. Prevention of ECM protein signaling may also prevent LEC metaplasia.

A surface modified IOL may also prevent LEC accumulation and Elschnig's pearl formation by inhibiting cellular growth. That is, by covalently attaching an RGD mimetic, RGD peptide, or flavonoid onto the surface of an acrylic lens, or an IOL made of a different material, the bioactivity bonding of the IOL surface will be enhanced so that the surface of the IOL will have the ability to attach LECs and form a monolayer between the lens capsule and the IOL surface. Because both sides of the cell will have a biological attachment, cell growth will be retarded. Inhibition of growth may be mediated by bioactive bonding or by a physiochemical mechanism that does not involve an intracellular mechanism.

### Methods for Surface Modifying Capsular Tension Rings

As explained in more detail below, two additional methods according to the invention involve surface modifying an exterior surface of a capsular tension ring (“tension ring”) with a mitotic inhibitor by covalently attaching the mitotic inhibitor to the exterior surface of the ring or modifying the exterior surface by coating it with a charged polyethylamine. The term “exterior surface” refers to the entire exterior or outer surface of the tension ring, that is, the surface which may be observed.

The inhibitor tension ring is then implanted into the capsular bag of an eye of a patient during extracapsular cataract surgery to act as a barrier inhibitor surface. In one method, the mitotic inhibitor is then available for killing reproducing (dividing) epithelial lens cells in the equatorial region after the surgery, thus preventing their proliferation and subsequent migration onto the posterior portion of the lens capsule. In an alternative method, the polyethylamine prevents the growth of lens epithelial cells on the surface of the tension ring allowing the ring to act as a barrier to the spread of the reproducing endothelial cells. The treated capsular tension rings are thus effective because they prevent multiplication or replication of the lens epithelial cells in contact with their surfaces in the equatorial region of the lens capsule and therefore prevent reproduction and migration of new lens epithelial cells toward the central area of the posterior lens capsule.

The first step in one method involves surface modifying the exterior surface of a capsular tension ring with an appropriate mitotic inhibitor. Preferably, the mitotic inhibitor comprises: (a) a conjugate of a cytotoxin or cytotoxic agent for destroying proliferating (reproducing) lens epithelial cells and preventing their eventual differentiation into fibroblasts; and (b) a spacer molecule. The spacer or extender molecule interfaces between the tension ring and the cytotoxin. While a spacer molecular is not required for attaching the conjugate to the ring surface, a spacer may be beneficial in providing the conjugate with flexibility to effectively enter the migrating cells near or on the treated surface. If the spacer is an anticollagen, as described below, it may be used to target the conjugate that might dissociate from the ring surface to the epithelial cells.

Preferably, the cytotoxin will be retained in the equatorial region of the capsular bag for a sufficient time to effectively destroy the dividing cells, which would have caused posterior lens capsule opacification. Although other cytotoxins may be used in accordance with the teachings of the present invention, the most preferred cytotoxin is methotrexate, which has been demonstrated in U.S. Pat. No. 4,515,794 to be effective in destroying the epithelial cells of the lens capsule which remain after cataract surgery. Methotrexate is well known in the art and is commercially available, for example, from Sigma Corporation.

Preferably, the cytotoxin is conjugated with a bovine serum albumin as a spacer or extender molecule. However, other proteins, biological materials, and non-biological materials which can interface between the tension ring and the cytotoxin, including, without limitation, chondroitin sulfate, polysaccharides, and short chain polymers, would also be within the scope of this method.

As previously described, any shape or design of capsular tension ring known in the art or to be developed may be utilized as the tension ring in the method of the invention. Preferably, the capsular tension ring is formed from PMMA. However, it is also within the scope of the invention to utilize another inert plastic material which is known or to be developed and which would exhibit similar properties as PMMA and be able to exert the necessary tension on the capsular bag. For example, formulations containing other acrylic segments, such as copolymers of HEMA and MMA would also be appropriate. Other inert materials, such as poly tetrafluoroethylene, may also be used in the construction and manufacture of the capsular tension ring according to the invention. Some flexibility may be achieved by the design and diameter of the capsular tension ring. That is, flexibility may result from the thinness of the tension ring or from the introduction of flexible chain segments. Such chain segments may be short polymer chains or repeat units that can rotate freely to impart flexibility to the main polymer chains.

The mitotic inhibitor may be covalently attached as a monolayer onto the capsular tension ring by any method known in the art or to be developed. In a preferred method, a covalent conjugate of MTX with bovine serum albumin may be attached to a PMMA capsular tension ring which has been plasma pretreated with ethylene diamine using glutaraldehyde (GA) to attach the conjugate to the treated PMMA lens.

More specifically, surface chemical activation of a capsular tension ring is performed using radio frequency (RF) plasma as the primary energy source, which first removes surface contaminants and etches the surface of the capsular tension ring by breaking and re-forming covalent bonds via short-lived free radicals. Subsequently, an amine-containing vapor, such as ethylenediamine or allylamine, is introduced into the plasma chamber to graft amino groups to the surface of the capsular tension ring. Following treatment with ethylenediamine, for example, the capsular tension ring is treated with glutaraldehyde, carbodiimide, or a similar compound, which functions as a linking agent to connect the amino groups on the tension ring to the spacer compound of the mitotic inhibitor. Finally, the capsular tension ring is incubated in an aqueous solution containing the desired mitotic inhibitor, such as a methotrexate-protein conjugate in a preferred embodiment. The resulting capsular tension ring thus has the mitotic inhibitor covalently bonded to its exterior surface.

As noted above, this method of treatment is intended to be exemplary, and alternative methods for coating the capsular tension ring which are known in the art or to be developed would also be appropriate. Exemplary methods of surface modification are described in, for example, U.S. Pat. Nos. 5,080,924; 5,260,093; 5,326,584; and 5,578,079, described previously.

After the capsular tension ring has been coated with the mitotic inhibitor to form an “inhibitor capsular tension ring,” the inhibitor capsular tension ring is implanted into a capsular bag of a patient during extracapsular cataract surgery using known methods of implantation. A schematic diagram of a capsular tension ring implanted in a capsular bag is shown in FIG. 1.

A further method according to the invention involves surface modifying an exterior surface of a capsular tension ring, as previously described, with a charged polyethylamine, a polymeric material capable of inducing cell death or preventing cell reproduction. Such materials are described, for example, in Proc. Natl. Acad. Sci. USA; 103; 17667 (2006), and are well known to possess antibacterial, antiviral, and antifungal properties. These materials, which have been shown to stand up from a surface in spikes, thus act as a cytotoxic barrier to cell growth on the surface of the capsular tension ring.

Preferred polyethylamine polymers for use in the invention have formula (1), wherein R₁ may be hydrogen or lower (C₁ to C₆) alkyl, R₂ may be C₁ to C₂₀ alkyl, and n is selected such that the molecular weight is about 500 to about 50,000 Daltons, depending on the particular ethyleneimine and the reaction conditions. The tertiary polyethylamine polymer may be reacted with an alkylating agent in a polar or non-polar solvent, such as ethanol or chloroform, to yield the quaternary nitrogen derivative of the polyethylamine polymer. For example, methyl iodide may be used as the alkylating agent when R₁=methyl. The preparation of polyethylamine polymers having formula (1) is described in Proc. Natl. Acad. Sci. USA; 103; 17667 (2006), for example.

—(N⁺R₁R₂—CH₂CH₂)ₙ (1)

Preferably, the coating may be applied to the capsular tension ring by dissolving the polyethylamine in an organic solvent to form a polymer solution, and then wiping, spraying, or painting the solution onto the medical device, or dipping the medical device into the solution. Appropriate organic solvents include alcohols (such as methanol, ethanol, propanol, and butanol), chlorinated hydrocarbons (such as chloroform), and esters (such as ethyl acetate). The solvent is then evaporated, leaving the polymer adhered to the surface of the tension ring. Alternatively, the surface of the tension ring may be functionalized with amino groups via RF-plasma treatment with amine-containing vapors, as previously described, and then treated with an appropriate ethyleneimine to form a polymeric coating chemically bonded to the surface of the capsular tension ring as a monolayer. It is also within the scope of the invention to impregnate the charged polyethylamine into the plastic or other material which comprises the CTR.

The resulting inhibitor capsular tension ring which is coated with the charged polyethylamine is then implanted into a capsular bag of an eye of a patient during extracapsular cataract surgery, as previously described.

It is also within the scope of the invention to apply the method of surface modification with a charged polyethylamine to the modification of the surfaces of intraocular lenses. The polyethylamine may be coated onto the IOL or may be copolymerized directly onto the lens, as previously described. The resulting coated IOL may then be implanted into a capsular bag of an eye of a patient during extracapsular lens surgery. The charged polyethylamine coating, which is capable of inducing cell death or preventing cellular reproduction, will provide antibacterial, antifungal, and antiviral properties to the IOL and will thus act as a cytotoxic barrier which prevents the growth of LECs on its surfaces. The coating will thereby inhibit the proliferation and/or migration of reproducing LECs and block the accumulation of LECs on the central area of the posterior capsule.

Without wishing to be bound by theory, it is believed that a tension ring having a surface treated with a mitotic inhibitor/cytotoxin to LECs will prevent and/or reduce LEC accumulation at the posterior capsule. That is, attachment of a mitotic inhibitor to the tension ring creates a barrier surface that should prevent residual anterior and equatorial LECs from proliferating across or behind the tension ring and accumulating at the posterior capsule. The mitotic inhibitor-modified capsular tension ring will come in contact with the residual reproducing and proliferating equatorial LECs and is expected to prevent opacification by blocking cellular migration leading to Elschnig's pearl formation, LEC metaplasia, or LEC accumulation across the tension ring.

Similarly, a capsular tension ring or IOL coated or treated with a charged polyethylamine, which is capable of inducing cell death and/or preventing cellular reproduction, will provide antibacterial, antifungal, and antiviral properties to the tension ring and will thus act as a cytotoxic barrier which prevents the growth of LECs on its surface. Therefore, LECs will be prevented from proliferating across or behind the tension ring and accumulating at the posterior capsule.

The claimed methods of implanting surface-modified inhibitor implantable ocular devices are advantageous and attractive because they are consistent with current cataract surgery processes (i.e., do not add additional steps), only affect actively dividing and/or migrating cells, and reduce the risk that the inhibitor or polyethylamine will damage cells other than LECs. Further, surface modification of an IOL does not result in changes in the optical characteristics and optical quality of the IOL or its mechanical properties. Similarly, surface modification of a capsular tension ring does not change its effectiveness or utility during cataract surgery. It is believed that implantation of a surface inhibitor modified inhibitor IOL according to the invention into the capsular bag of the eye will result in inhibition of LEC migration and accumulation onto the posterior capsule. Similarly, it is believed that implantation of a surface modified inhibitor capsular tension ring into the capsular bag of the eye will result in inhibition of LECs, Elschnig's pearls, and fibrosis on the posterior lens capsule. This inhibition is mediated by the modified tension ring.

### Implantable Ocular Devices

This invention also relates to an inhibitor implantable ocular device which comprises a substrate having a surface, a first chemical moiety grafted onto the surface, and a first non-cytotoxic inhibitor compound covalently bonded to the chemical moiety.

In one embodiment, the implantable ocular device is an intraocular lens. As previously explained, the preferred substrates for IOLs include materials such as a silicone, a soft acrylic, such as HEMA, or a hard acrylic, such as PMMA. However, it is also within the scope of the invention to utilize another inert, optically clear plastic material which is known or to be developed and which would exhibit similar properties to these exemplified polymers.

Also, as previously described, the IOL may be of any shape or design known in the art or to be developed, including IOLs containing at least one haptic, most typically two haptics which are situated about 180° apart. Such haptics may be of any type, material, number, or configuration known in the art or to be developed, and may be of the same material as the IOL or of a different material. Any type of haptics which are known in the art or to be developed may be components of the IOL according to the invention. Appropriate chemical moieties and inhibitor compounds for attachment to the IOL have been previously described. In one embodiment, when the IOL contains haptics, the surfaces of the haptics may contain (second) chemical moieties grafted onto the surfaces and (second) non-cytotoxic inhibitor compounds covalently bonded to the chemical moieties. Preferably, the chemical moieties and inhibitor compounds which are attached to the surfaces of the haptics are identical to those attached to the surface of the IOL. That is, the first and second chemical moieties are the same, and the first and second inhibitor compounds are the same. When implanted into an eye of a patient during extracapsular cataract surgery, the implantable ocular device according to the invention prevents, minimizes, or delays the formation of posterior capsule opacification.

In a second preferred embodiment, the implantable ocular device is a capsular tension ring. As previously explained, the preferred substrate for a capsular tension ring is PMMA, but other inert polymers which are known in the art or to be developed which would provide similar properties to PMMA and exert an appropriate level of tension in the capsular bag would also be appropriate. Also, as previously described, the capsular tension ring may be of any shape or design known in the art or to be developed, including tension rings of varying diameters and thickness and rings containing fixation hook(s) or suturing hole(s). Tension rings according to the invention may be closed rings, foldable ring, partial rings, segmental rings, and rings having circumferences of any degree, including rings which span 270°, rings which span 360° or nearly 360°, and all rings which span intermediate angles. Appropriate chemical moieties and inhibitor compounds for attachment to the tension ring have been previously described. When implanted into an eye of a patient during extracapsular cataract surgery, the capsular tension ring according to the invention prevents, minimizes, or delays the formation of posterior capsule opacification.

This invention also relates to a first embodiment of a second type of inhibitor capsular tension ring, which comprises a substrate having an exterior surface and a selected mitotic inhibitor covalently attached to the exterior surface. As previously explained, the preferred substrate is PMMA, but other inert polymers (known in the art or to be developed) which would provide similar properties to PMMA and exert an appropriate level of tension in the capsular bag would also be appropriate. Also, as previously described, the capsular tension ring may be of any shape or design known in the art or to be developed, including tension rings of various diameters and thicknesses and rings containing fixation hook(s) or suturing hole(s). Tension rings according to the invention may be closed rings, foldable rings, partial rings, segmental rings, and rings having circumferences of any degree, including rings which span 270°, rings which span 360° or nearly 360°, and all rings which span intermediate angles. Appropriate and preferred targeted mitotic inhibitor compounds for coating on the tension ring have been previously described, but the most preferred mitotic inhibitor comprises a conjugate of MTX and a bovine serum albumin. When implanted into an eye of a patient during extracapsular cataract surgery, the capsular tension ring according to this first embodiment prevents, minimizes, or delays the formation of posterior capsule opacification.

A second embodiment of a second type of inhibitor capsular tension ring according to the invention comprises a substrate having an exterior surface and a charged polyethylamine coated on the exterior surface. Appropriate and preferred materials, shapes, and designs for the capsular tension ring have been previously described. Preferably, the polyethylamine has formula (1), described above. When implanted into an eye of a patient during extracapsular cataract surgery, the capsular tension ring according to this second embodiment prevents, minimizes, or delays the formation of posterior capsule opacification.

The inventive methods of surface modifying capsular tension rings with a mitotic inhibitor or charged polyethylamine and the treated capsular tension rings according to the invention are superior to the known methods of physically applying coatings to intraocular lenses and the resulting coated IOLs for two reasons. First, the known coated IOLs do not contain chemical bonds between the coating material and the lens, and thus the coatings according to the embodiments of the present invention are more stable due to the strong covalent attachment of the coatings to the tension rings. Further, the prior art physical interaction of mitotic inhibitor and IOL is a reversible interaction and thus the mitotic inhibitor may be released and cause toxicity. In contrast, surface modification of the mitotic inhibitor with the capsular tension ring via covalent bonds is essentially an irreversible reaction and will greatly reduce the risk of toxicity. Additionally, coating intraocular lenses does not affect the peripheral reproducing LECs, but rather primarily halts the migration of the LECs across the posterior capsule under the IOL. In contrast, the present methods of coating capsular tension rings address the problem at its source. That is, by targeting the initial reproducing LECs, cell proliferation is stopped very early, before migration starts, which is advantageous.

The invention will now be illustrated in connection with the following, non-limiting examples.

### Example 1

### Modification of Acrylic IOL Surface

An acrylic IOL material is washed with an aqueous sodium dodecyl sulfate solution in an ultrasonic water bath to remove any contaminants, such as dirt and dust, which might be present on the IOL surface. The surface chemistry of the acrylic IOL is then determined using Attenuated Total Reflection Fourier Transform Infra-Red Spectroscopy (ATR-FTIR) in order to establish a chemical baseline signal that will allow measurement of the changes in the surface chemical functionality which is introduced by the subsequent argon plasma treatment. The IOL is also examined microscopically using an environment scanning electron microscope to establish the initial conditions of the surface morphology prior to the argon plasma treatment.

The IOL is then placed in an RF-plasma reactor and treated first with argon plasma and then grafted with an appropriate chemical moiety. Specifically, grafting is accomplished by the introduction of a selected organic vapor into the plasma reactor in order to graft the specific chemical moiety (e.g., acrylic acid to produce carboxyl groups). The presence of these carboxyl groups is confirmed by comparing the chemical profiles of the untreated and the plasma grafted IOLs using FTIR. Such groups will then be used to covalently link the desired inhibitor to the IOL surface using an appropriate catalyst.

The chemical presence of the inhibitor compound on the surface of the IOL is also examined using Electron Spectroscopy for Chemical Analysis (ESCA), which determines the amount of the unique chemical group or element immobilized on the IOL by identifying its specific binding energy.

### Example 2

### Covalent Attachment of Inhibitor Compound to the IOL

The acrylic IOL with carboxyl groups immobilized on its surface is covalently attached to the desired inhibitor compound. The protocol for covalent attachment depends on the functional groups present on the selected compound. For example, if the selected compound contains amino groups (i.e., an RGD mimetic or RGD peptide), the acrylic acid-grafted IOL is treated with a water-soluble carbodiimide to activate the carboxyl groups on the IOL and to facilitate its reaction with the amino group. A wash step using distilled water at room temperature is typically performed. The IOL is incubated in a pH-adjusted solution of the selected compound to allow the amino groups of the compound to react with the activated carboxyl groups on the surface to form amide bonds. Immobilization of compounds on PMMA has previously been demonstrated to be pH dependent (Kang; Biomaterial; 14:787-792 (1993)). Thus, the inhibitor compound is coupled onto the acrylic IOL at various pH values (3, 7, and 11) to promote immobilization.

For covalently attaching flavonoids, which contain hydroxyl groups, the acrylic acid-grafted IOL is treated with oxalyl chloride to convert the COOH groups to the highly reactive corresponding acid chloride. A wash or rinse with a non-protic solvent, such as methylene chloride or ethyl ether, may be desirable to remove excess oxalyl chloride. The COCL groups then react with hydroxyl groups on the flavonoids to form esters.

The presence and stability of the covalently bound inhibitor compounds on the IOL are confirmed by ESCA.

### Example 3

### Evaluation of Modified IOL

Following modification, changes in chemical stability and surface roughness of the modified IOL are determined as follows. The modified IOL is incubated in de-ionized water at 45° C., a temperature capable of breaking secondary bonds. When compared to the spectra before incubation, FTIR analysis is used to verify the presence of the introduced functional groups which remain after incubation. Specifically, the presence of carbonyl groups indicates that the flavonoid, RGD mimetic, or RGD peptide is covalently attached to the surface of the acrylic IOL and that the bond formation is stable. Similarly, ESCA analysis may also be used to confirm immobilization of these compounds onto the IOL. Peaks corresponding to the carbonyl and phenol groups from the ESCA survey scan spectrum will indicate that a flavonoid, RGD mimetic, or RGD peptide is covalently attached to the IOL. Changes in the ESCA spectrum will also identify if any chemical moieties, important for inhibition, were blocked or disrupted during the covalent crosslinking based on changes of its signal.

The surface morphology of the modified IOL is evaluated by scanning electron microscopy to confirm that there are no major morphological changes on the surface of the IOL, such as excessive roughness that could lead to opacity, which would indicate that the IOL may be damaged and unsuitable for implantation. The most preferred modified IOL will demonstrate stable covalent bond formation, little to no changes in surface morphology, and the presence of biologically active molecular attachments (i.e., RGD mimetics, RGD peptides, or flavonoids).

### Example 4

### Evaluation of Inhibition of LEC Accumulation

Evaluation of the ability of the surface-modified acrylic IOL to inhibit LEC accumulation is performed using the in vitro capsular bag model, which is considered to be a well-established model for PCO studies (Liu; Invest. Opthalmol. Vis. Sci; 37:906-914 (1996)). The modified IOL is implanted into the capsular bag of a porcine eye globe obtained from a slaughterhouse. The capsular bag is then dissected, pinned on a plastic culture dish, and cultured in supplemented minimal essential medium.

Accumulation of LECs is evaluated for up to 4 weeks in culture using phase contrast microscopy. LEC growth is evaluated at days 2, 4, 7, 10, 14, 21, and 28. A graticroscope eyepiece is used to determine the total area of posterior capsule coverage and the total area of capsule coverage is calculated by determining the number of squares covered by LECs within the graticule at different times in culture. Cell proliferation is also measured via fluorescence microscopy using the LIVE/DEAD cell proliferation assay (Invitrogen). Grafted acrylic IOLs, exposed to the inhibitory compound in the absence of the carbodiimide, and untreated IOL will serve as negative controls, that is, IOLs that are not expected to exhibit inhibition.

A significant reduction of LEC accumulation on the posterior lens capsule indicates that the compound immobilized on the surface of the acrylic IOL has successfully inhibited proliferation and/or migration of LECs cells.

### Example 5

### Modification of PMMA Capsular Tension Ring Surface

A PMMA capsular tension ring material is washed with an aqueous sodium dodecyl sulfate solution in an ultrasonic water bath to remove contaminants, such as dirt and dust, which are present on the tension ring. The surface chemistry of the acrylic tension ring is then determined using Attenuated Total Reflection Fourier Transform Infra-Red Spectroscopy (ATR-FTIR) in order to establish a chemical baseline signal that will allow measurement of the changes in the surface chemical functionality introduced by the subsequent grafting using argon plasma. The tension ring is also examined microscopically using an environment scanning electron microscope to establish the initial conditions of the surface morphology prior to the argon plasma treatment.

The tension ring is then placed in an RF-plasma reactor and treated first with argon plasma and then grafted with an appropriate chemical moiety. Specifically, grafting is accomplished by the introduction of a selected organic vapor into the plasma reactor in order to graft the specific chemical moiety (e.g., acrylic acid to produce carboxyl groups). The presence of these carboxyl groups is confirmed by comparing the chemical profiles of the untreated and the plasma grafted tension rings using FTIR. Such groups will then be used to covalently crosslink the desired inhibitor to the tension ring surface using an appropriate catalyst.

The chemical presence of the inhibitor compound on the surface of the tension ring is also examined using ESCA.

### Example 6

### Covalent Attachment of Inhibitor Compound to the Tension Ring

The carboxyl groups immobilized on the PMMA tension ring are covalently attached to the desired inhibitor compound. The protocol for covalent attachment depends on the functional groups present on the selected compound. If the selected compound contains amino groups (i.e., an RGD mimetic or RGD peptide), the acrylic acid grafted tension ring is treated with a water-soluble carbodiimide to activate the carboxyl groups on the tension ring. A wash step using distilled water at room temperature is typically performed. The tension ring is incubated in a pH-adjusted solution of the selected compound to allow the amino groups of the compound to react with the carboxyl groups on the activated surface to form amide bonds. Immobilization of compounds on PMMA has previously been demonstrated to be pH dependent (Kang (Biomaterial; 14:787-792 (1993))). Thus, the inhibitor compound is coupled onto the acrylic tension ring at various pH values (3, 7, and 11) to promote immobilization.

For covalently attaching flavonoids, which contain hydroxyl groups, the acrylic acid-grafted tension ring is treated with oxalyl chloride to convert the COOH groups to the highly reactive corresponding acid chloride. A wash or rinse with a non-protic solvent, such as methylene chloride or ethyl ether, may be desirable to remove excess oxalyl chloride. The COCL groups then react with hydroxyl groups on the flavonoids to form esters.

The presence and stability of the covalently-linked inhibitor compounds on the tension ring are measured by examining the binding energy of a characteristic chemical group or element of the respective compound, such as a sulfur-containing group, using ESCA.

### Example 7

### Evaluation of Modified Capsular Tension Ring

Following modification, changes in chemical stability and surface roughness of the modified tension ring are determined as follows. The modified tension ring is incubated in de-ionized water at 45° C., a temperature capable of breaking secondary bonds. When compared to the spectra before incubation, FTIR analysis is used to verify the presence of the introduced functional groups which remain after incubation. Specifically, the presence of carbonyl groups indicates that the flavonoid, RGD mimetic, or RGD peptide is covalently attached to the surface of the tension ring and that the bond formation is stable. ESCA analysis is also used to confirm immobilization of the compound onto the tension ring. Peaks corresponding to the carbonyl and phenol groups from the ESCA survey scan spectrum will indicate that a flavonoid, RGD mimetic, or RGD peptide is covalently attached to the tension ring. Changes in the ESCA spectrum will also identify if any chemical moieties, important for inhibition, were blocked or disrupted during the covalent linking based on changes of its signal.

The surface morphology of the modified tension ring is evaluated by scanning electron microscopy to confirm that there are no major morphological changes on the surface of the tension ting, such as excessive roughness that could interfere with the efficiency of the tension ring and make it unsuitable for implantation. The most preferred modified tension ring will demonstrate stable covalent bond formation, little to no changes in surface morphology, and the presence of biologically molecular attachments (i.e., RGD mimetics, RGD peptides, or flavonoids).

### Example 8

### Evaluation of Inhibition of LEC Accumulation

Evaluation of the ability of the surface-modified acrylic tension ring to inhibit LEC accumulation is performed using the in vitro capsular bag model, which is considered to be a well-established model for PCO studies (Liu; Invest. Ophthalmol. Vis. Sci; 37:906-914 (1996)). The modified tension ring is implanted into the capsular bag of a porcine eye globe obtained from a slaughterhouse. The capsular bag is then dissected, pinned on a plastic culture dish, and cultured in supplemented minimal essential medium.

Accumulation of LECs using phase contrast microscopy is evaluated for up to 4 weeks in culture using phase contrast microscopy. LEC growth is evaluated at days 2, 4, 7, 10, 14, 21, and 28. A graticroscope eyepiece is used to determine the total area of posterior capsule coverage and the total area of capsule coverage is calculated by determining the number of squares covered by LECs within the graticule at different times in culture. Cell proliferation is also measured via fluorescence microscopy using the LIVE/DEAD cell proliferation assay (Invitrogen). Grafted PMMA tension rings, exposed to the inhibitory compound in the absence of the carbodiimide, and untreated tension ring will serve as negative controls, that is, tension rings that are not expected to exhibit inhibition.

A significant reduction of LEC accumulation to the posterior lens capsule indicates that the compound immobilized on the surface of the acrylic tension ring has successfully inhibited proliferation and/or migration of LECs cells.

### Example 9

### Preparation of Inhibitor Capsular Tension Ring

A PMMA capsular tension ring material is washed with an aqueous sodium dodecyl sulfate solution in an ultrasonic water bath to remove contaminants, such as dirt and dust, which are present on the tension ring. The surface chemistry of the acrylic tension ring is then determined using ATR-FTIR in order to establish a chemical baseline signal that will allow measurement of the changes in the surface chemical functionality introduced by the subsequent graft-induced argon plasma treatment. The tension ring is also examined microscopically using a scanning electron microscope to establish the initial conditions of the surface morphology prior to the argon plasma treatment.

The tension ring is then placed in an RF-plasma reactor and treated first with argon plasma and then grafted with chemically reactive amino groups by introducing ethylenediamine into the plasma reactor. The presence of these amino groups is confirmed by comparing the chemical profiles of the untreated and the plasma grafted tension rings using FTIR.

The tension ring is then further treated with glutaraldehyde (GA), rinsed of excess GA, and then treated with an MTX-protein conjugate. The conjugate is a covalent conjugate of MTX with bovine serum albumin prepared using ECDI as the condensing agent, by the procedure of Kulkarni et al., Cancer Res. 41; 2700 (1981). Control tension rings are treated with unconjugated protein (no MTX).

The chemical presence of the mitotic inhibitor on the surface of the tension ring is examined using ESCA.

### Example 10

### Evaluation of Inhibitor Capsular Tension Ring

Treated slides prepared using the same method described in EXAMPLE 9 were evaluated for their ability to support/inhibit the outgrowth of lens epithelial cells from bovine anterior capsules. Outgrowth was followed for six days. It was found that the slides treated with the covalent conjugate of MTX inhibited cell outgrowth. When these slides were rinsed and evaluated again, they inhibited outgrowth for a second set of lens capsules. A third set of cells was somewhat inhibited, but not completely. This inhibition was not due to the glutaraldehyde, since the protein (alone)-treated slide allowed good outgrowth. Further, an MTX (alone)-treated slide inhibited outgrowth during the first evaluation period, but not during subsequent evaluations.

### Example 11

### Evaluation of Inhibitor Capsular Tension Ring

Evaluation of the ability of the surface-modified acrylic tension ring to inhibit LEC accumulation is performed using the in vitro capsular bag model, which is considered to be a well-established model for PCO studies (Liu; Invest. Ophthalmol. Vis. Sci; 37:906-914 (1996)). The treated tension ring is implanted into the capsular bag of a porcine eye globe obtained from a slaughterhouse. The capsular bag is then dissected, pinned on a plastic culture dish, and cultured in supplemented minimal essential medium.

Accumulation of LECs using phase contrast microscopy is evaluated for up to 4 weeks in culture using phase contrast microscopy. LEC growth is evaluated at days 2, 4, 7, 10, 14, 21, and 28. A graticule eyepiece is used to determine the total area of posterior capsule coverage, and the total area of capsule coverage is calculated by determining the number of squares covered by LECs within the graticule at different times in culture. Cell proliferation is also measured via fluorescence microscopy using the LIVE/DEAD cell proliferation assay (Invitrogen). PMMA tension rings treated with unconjugated protein or MTX and untreated tension ring will serve as negative controls, that is, tension rings that are not expected to exhibit inhibition.

A significant reduction of LEC accumulation to the posterior lens capsule indicates that the compound immobilized on the surface of the acrylic tension ring has successfully inhibited proliferation and/or migration of LECs cells.

### Example 12

### Preparation of Capsular Tension Ring Coated with Polyethylamine

Linear N,N-dodecylmethylpolyethylamine is prepared as described in Proc. Natl. Acad. Sci. USA; 103; 17667 (2006) and dissolved in butanol to form a solution. A capsular tension ring is coated with the solution and the solvent is evaporated to yield a tension ring coated with N,N-dodecylmethylpolyethylamine.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for preventing, minimizing, or delaying posterior capsule opacification, the method comprising:
(a) chemically activating a surface of an implantable ocular device by grafting a first chemical moiety onto the surface of the device;
(b) covalently attaching a first non-cytotoxic inhibitor compound to the first chemical moiety on the chemically activated surface of the implantable ocular device to produce an inhibitor implantable ocular device; and
(c) implanting the inhibitor implantable ocular device into a capsular bag of an eye of a patient during extracapsular cataract surgery.

2. The method according to claim 1, wherein the first chemical moiety is an amino or a carboxyl group.

3. The method according to claim 1, wherein the first inhibitor compound contains at least one functional group selected from the group consisting of an amino group, a hydroxyl group, and a carboxyl group.

4. The method according to claim 1, wherein the first inhibitor compound is selected from the group consisting of a flavonoid, an RGD mimetic, and an RGD peptide.

5. The method according to claim 1, wherein the implantable ocular device is selected from the group consisting of an intraocular lens and a capsular tension ring.

6. The method according to claim 5, wherein the implantable ocular device comprises an intraocular lens, and wherein the device further comprises at least one haptic having a surface, the method further comprising at least before step (c):
(b′) chemically activating the surface of the at least one haptic by grafting a second chemical moiety onto the surface of the at least one haptic; and
(b″) covalently attaching a second non-cytotoxic inhibitor compound to the second chemical moiety on the chemically activated surface of the at least one haptic.

7. The method according to claim 6, wherein steps (b′) and (b″) are performed substantially simultaneously with steps (a) and (b).

8. An implantable ocular device comprising: a substrate having a surface, a first chemical moiety grafted onto the surface, and a first non-cytotoxic inhibitor compound covalently bonded to the first chemical moiety, wherein the implantable ocular device prevents, minimizes, or delays the formation of posterior capsule opacification when implanted into an eye of a patient during extracapsular cataract surgery.

9. The implantable ocular device according to claim 8, wherein the first chemical moiety is an amino group or a carboxyl group.

10. The implantable ocular device according to claim 8, wherein the first inhibitor compound contains at least one functional group selected from the group consisting of an amino group, a hydroxyl group, and a carboxyl group.

11. The implantable ocular device according to claim 8, wherein the first inhibitor compound is selected from the group consisting of a flavonoid, an RGD mimetic, and an RGD peptide.

12. The implantable ocular device according to claim 8, wherein the device is selected from the group consisting of an intraocular lens and a capsular tension ring.

13. The implantable ocular device according to claim 12, wherein the device comprises an intraocular lens, further comprising at least one haptic having a surface, a second chemical moiety grafted onto the surface of the at least one haptic, and a second non-cytotoxic inhibitor compound covalently bonded to the second chemical moiety on the surface of the at least one haptic.

14. A method for preventing, minimizing or delaying posterior capsule opacification, the method comprising:
(a) surface modifying an exterior surface of a capsular tension ring by covalently attaching a mitotic inhibitor to the exterior surface to produce an inhibitor capsular tension ring; and
(b) implanting the inhibitor capsular tension ring into a capsular bag of an eye of a patient during extracapsular cataract surgery.

15. The method according to claim 14, wherein the mitotic inhibitor comprises a conjugate of a cytotoxic agent and a spacer molecule.

16. The method according to claim 15, wherein the spacer molecule comprises a protein.

17. The method according to claim 15, wherein the cytotoxic agent comprises a cytotoxin to lens epithelial cells.

18. The method according to claim 17, wherein the cytotoxin comprises methotrexate.

19. The method according to claim 14, wherein step (a) comprises grafting amino groups onto the exterior surface of the capsular tension ring by treating the capsular tension ring with radio frequency plasma and an amine-containing vapor, treating the capsular tension ring with a linking agent, and treating the capsular tension ring with the mitotic inhibitor, wherein the linking agent binds the amino groups on the exterior surface of the capsular tension ring to the mitotic inhibitor.

20. A capsular tension ring comprising a substrate having an exterior surface and a mitotic inhibitor covalently attached to the surface, wherein the capsular tension ring prevents, minimizes, or delays the formation of posterior capsule opacification when implanted into an eye of a patient during extracapsular cataract surgery.

21. The capsular tension ring according to claim 20, wherein the mitotic inhibitor comprises a conjugate of a cytotoxic agent and a spacer molecule.

22. The capsular tension ring according to claim 21, wherein the spacer molecule comprises a protein.

23. The capsular tension ring according to claim 21, wherein the cytotoxic agent comprises a cytotoxin to lens epithelial cells.

24. The capsular tension ring according to claim 23, wherein the cytotoxin comprises methotrexate.

25. A method for preventing, minimizing or delaying posterior capsule opacification, the method comprising:
(a) surface modifying an exterior surface of a capsular tension ring by coating or grafting the exterior surface with a charged polyethylamine to produce an inhibitor capsular tension ring; and
(b) implanting the inhibitor capsular tension ring into a capsular bag of an eye of a patient during extracapsular cataract surgery.

26. The method according to claim 25, wherein the charged polyethylamine has formula (1), wherein R₁=H or C₁ to C₆ alkyl, R₂=C₁ to C₂₀ alkyl, and n is selected to provide a molecular weight of about 500 to about 50,000 Daltons.
—(N⁺R₁R₂—CH₂CH₂)ₙ (1)

27. The method according to claim 25, wherein step (a) comprises grafting amino groups onto the exterior surface of the capsular tension ring by treating the capsular tension ring with radio frequency plasma and an amine-containing vapor followed by an ethyleneimine vapor to produce a polyethylamine grafted coating chemically bonded to the exterior surface of the capsular tension ring.

28. The method according to claim 25, wherein step (a) comprises dissolving the polyethylamine in an organic solvent to yield a polymer solution; spraying, wiping, or painting the polymer solution on the exterior surface of the capsular tension ring; and evaporating the organic solvent to form a polyethylamine coating on the exterior surface of the capsular tension ring.

29. A capsular tension ring comprising a substrate having an exterior surface and a charged polyethylamine coated on the exterior surface, wherein the capsular tension ring prevents, minimizes, or delays the formation of posterior capsule opacification when implanted into an eye of a patient during extracapsular cataract surgery.

30. The capsular tension ring according to claim 29, wherein the charged polyethylamine has formula (1), wherein R₁=H or C₁ to C₆ alkyl, R₂=Cᵢ to C₂₀, and n is selected to provide a molecular weight of about 500 to about 50,000 Daltons.
—(N⁺R₁R₂—CH₂CH₂)ₙ (1)
